(19) United States
(12) Patent Application Publication
(10) Pub. No.: US 2021/0196240 A1
(43) Pub. Date: Jul. 1, 2021

(54) ULTRASONIC DIAGNOSTIC APPARATUS AND PROGRAM FOR CONTROLLING THE SAME

(71) Applicant: General Electric Company, Schenectady, NY (US)

(72) Inventors: Naohisa KAMIYAMA, Tokyo (JP); Takuma OGURI, Tokyo (JP); Sayuka SAGA, Tokyo (JP)

(21) Appl. No.: 17/117,612

(22) Filed: Dec. 10, 2020

(30) Foreign Application Priority Data
Dec. 25, 2019 (JP) 2019-234161

(51) Int. Cl. A61B 8/00; A61B 8/14; A61B 8/08

(52) U.S. Cl. CPC A61B 8/54; A61B 8/14; A61B 8/469; A61B 8/463; A61B 8/5246

(57) ABSTRACT

Ultrasonic diagnostic apparatus capable of improving visibility of a low-brightness region in a B-mode image while maintaining visibility of a high-brightness region, a processor produces a first B-mode image and a second B-mode image BI2, locates a first region R1 in the first B-mode image having lower brightness than a surrounding region, and produces a combined image I in which an image BI2a of a second region R2 at a position in the second B-mode image BI2 corresponding to a position of the first region R1 is combined into the position of the first region R1 in the first B-mode image for display on a display 8. The first B-mode image is obtained using a first condition group, and the second B-mode image BI2 is obtained using a second condition group defined so that brightness of the second B-mode image BI2 is higher than that of the first B-mode image.

### FIELD

The present invention relates to an ultrasonic diagnostic apparatus for acquiring a B-mode image of a patient, and a method of controlling the same.

### BACKGROUND

Ultrasonic diagnostic apparatuses are used in examinations of a variety of body parts of a patient. For example, in a breast ultrasonic examination using an ultrasonic diagnostic apparatus, tissue properties of a mammary gland and its surrounding tissue, such as fat, are observed to study the presence/absence of lesion tissue (see WO2018/180386, for example). In many cases, the lesion tissue is rendered in a B-mode image with lower brightness than surrounding tissue, and benignancy/malignancy is decided by making a close examination of the position and shape of a low-brightness region.

In B-mode images, ruptures in a boundary (anterior boundary) between the mammary gland and fat tissue and a boundary (posterior boundary) between the mammary gland and pectoralis major muscle are an important observation for evaluating infiltration of a cancer to tissue other than the mammary gland. In conventional ultrasonic diagnostic apparatuses, however, visibility of the posterior boundary is sometimes significantly poorer than the anterior boundary due to an effect of attenuation in a tumor mass, etc., and improvement of visibility is desired.

Moreover, visibility of the inside of a tumor mass is also an important observation for making a diagnosis of massiveness specific to a cancer. Since the inside of many tumor masses is rendered with lower brightness than the mammary gland and/or fat tissue, and the gain is adjusted to the mammary gland and fat tissue other than the tumor mass in an ordinary ultrasonic examination, it is necessary to increase the gain for making a close observation of the inside of the tumor mass. However, it is extremely difficult to adjust the gain taking account of visibility for both the inside of the tumor mass and surrounding tissue at the same time.

From these circumstances, there is a need for an ultrasonic diagnostic apparatus capable of improving visibility of a low-brightness region while maintaining visibility of a high-brightness region in a B-mode image.

### BRIEF SUMMARY

This summary introduces concepts that are described in more detail in the detailed description. It should not be used to identify essential features of the claimed subject matter, nor to limit the scope of the claimed subject matter.

The invention, in one aspect thereof made for solving the aforementioned problem, is an ultrasonic diagnostic apparatus comprising: an ultrasonic probe for transmitting ultrasound to a patient and receiving echo signals for said ultrasound; one or more processors; and a display, wherein said one or more processors are configured to: control said ultrasonic probe to transmit said ultrasound, produce a first B-mode image and a second B-mode image based on echo signals for said ultrasound, said first B-mode image being obtained using a first condition group including a plurality of conditions, said second B-mode image being obtained using a second condition group including a plurality of conditions, said second condition group including a condition(s) with a property different from that of at least one of the plurality of conditions included in said first condition group, said second condition group being defined so that brightness of said second B-mode image is higher than that of said first B-mode image, locate a first region in said first B-mode image having lower brightness than a surrounding region, and produce a combined image in which an image of a second region at a position in said second B-mode image corresponding to a position of said first region is combined into the position of said first region in said first B-mode image for display on said display.

According to the invention in the aspect above, in the combined image, the image in the first region having lower brightness than a surrounding region in the first B-mode image is replaced with the second B-mode image having higher brightness than the first B-mode image, and therefore, visibility for the first region is improved. On the other hand, in regions other than the first region in the combined image, the first image is displayed, and therefore, visibility can be maintained there.

### BRIEF DESCRIPTION OF THE DRAWINGS

FIG. 1 is a block diagram showing an exemplary embodiment of a configuration of the ultrasonic diagnostic apparatus in the present invention;

FIG. 2 is a flow chart showing an example of processing in the ultrasonic diagnostic apparatus shown in FIG. 1;

FIG. 3 is a diagram showing a first B-mode image in which an acoustic shadow appears;

FIG. 4 is a diagram showing a second B-mode image;

FIG. 5 is a diagram showing examples of a first function and a second function used in logarithm compression processing;

FIG. 6 is a diagram explaining production of a combined image, and showing the combined image created; and

FIG. 7 is a diagram showing a combined image in which a color image is displayed.

### DETAILED DESCRIPTION

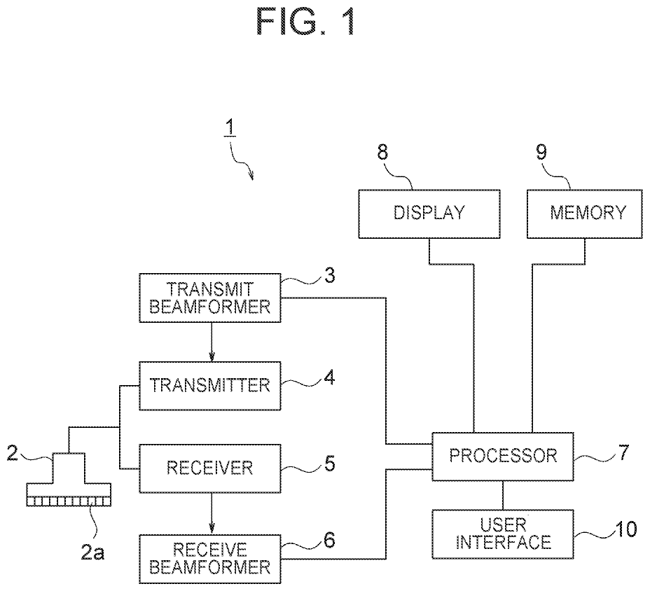

Embodiments of the present disclosure will now be described, by way of example, with reference to the Figures. An ultrasonic diagnostic apparatus 1 shown in FIG. 1 comprises a transmit beamformer 3 and a transmitter 4 for driving a plurality of vibrator elements 2a arranged in an ultrasonic probe 2 to emit pulsed ultrasonic signals to a patient (not shown). The pulsed ultrasonic signals are reflected in the inside of the patient to generate echoes that return to the vibrator elements 2a. The echoes are converted into electrical signals by the vibrator elements 2a, and the electrical signals are received by a receiver 5. The electrical signals representing the received echoes, i.e., echo signals, undergo amplification, etc. with a required gain at the receiver 5, and then input to a receive beamformer 6, where receive beamforming is performed thereon. The receive beamformer 6 outputs receive-beamformed ultrasound data.

The receive beamformer 6 may be a hardware beamformer or a software beamformer. In the case that the receive beamformer 6 is a software beamformer, it may comprise one or more processors including a graphics processing unit (GPU), a microprocessor, a central processing unit (CPU), a digital signal processor (DSP), or any one or more of other kinds of processors capable of executing logical operations. The processor(s) constituting the receive beamformer 6 may be constructed from a processor separate from a processor 7, which will be described later, or constructed from the processor 7.

The ultrasonic probe 2 may comprise electrical circuitry to perform all or part of the transmit and/or receive beamforming. For example, all or part of the transmit beamformer 3, transmitter 4, receiver 5, and receive beamformer 6 may be situated within the ultrasonic probe 2.

The ultrasonic diagnostic apparatus 1 also comprises the processor 7 for controlling the transmit beamformer 3, transmitter 4, receiver 5, and receive beamformer 6. The processor 7 is in electronic communication with the ultrasonic probe 2. The processor 7 may control the ultrasonic probe 2 to acquire ultrasound data. The processor 7 controls which of the vibrating elements 2a are active, and the shape of an ultrasonic beam transmitted from the ultrasonic probe 2. The processor 7 is also in electronic communication with the display 8, and the processor 7 may process the ultrasound data into ultrasonic images for display on the display 8. The phrase “electronic communication” may be defined to include both wired and wireless connections. The processor 7 may include a central processing unit (CPU) according to one embodiment. According to other embodiments, the processor 7 may include other electronic components capable of carrying out processing functions, such as a digital signal processor, a field-programmable gate array (FPGA), a graphics processing unit (GPU), or any other type of processor. According to other embodiments, the processor 7 may include a plurality of electronic components capable of carrying out processing functions. For example, the processor 7 may include two or more electronic components selected from a list of electronic components including: a central processing unit, a digital signal processor, a field-programmable gate array, and a graphics processing unit.

The processor 7 may also include a complex demodulator (not shown) that demodulates RF data. In another embodiment, the demodulation can be carried out earlier in the processing chain.

The processor 7 is adapted to perform one or more processing operations according to a plurality of selectable ultrasonic modalities on the data. The data may be processed in real-time during a scanning session as the echo signals are received. For the purpose of this disclosure, the term “real-time” is defined to include a procedure that is performed without any intentional delay.

The data may be temporarily stored in a buffer (not shown) during ultrasonic scanning, so that they can be processed in a live operation or in an off-line operation not in real-time. In this disclosure, the term “data” may be used in the present disclosure to refer to one or more datasets acquired with an ultrasonic apparatus.

The ultrasound data may be processed by other or different mode-related modules by the processor 7 (e.g., B-mode, color Doppler, M-mode, color M-mode, spectral Doppler, elastography, TVI, strain, strain rate, and the like) to form data for ultrasonic images. For example, one or more modules may produce ultrasonic images in B-mode, color Doppler, M-mode, color M-mode, spectral Doppler, elastography, TVI, strain, strain rate, and combinations thereof, and the like. The image beams and/or image frames are stored and timing information indicating a time at which the data was acquired in memory may be recorded. The modules may include, for example, a scan conversion module to perform scan conversion operations to convert the image frames from coordinate beam space to display space coordinates. A video processor module may be provided that reads the image frames from memory and displays the image frames in real-time while a procedure is being carried out on the patient. The video processor module may store the image frames in image memory, from which the ultrasonic images are read and displayed on the display 8.

The ultrasound data before the scan conversion operations will be referred to herein as raw data. The data after the scan conversion operations will be referred to herein as image data.

In the case that the processor 7 includes a plurality of processors, the aforementioned processing tasks to be handled by the processor 7 may be handled by the plurality of processors. For example, a first processor may be utilized to demodulate and decimate the RF signal while a second processor may be used to further process the data prior to displaying an image.

In the case that the receive beamformer 6 is a software beamformer, for example, its processing functions may be carried out by a single processor or by a plurality of processors.

The display 8 is an LCD (Liquid Crystal Display), an organic EL (Electro-Luminescence) display, or the like.

The memory 9 is any known data storage medium, and comprises non-transitory storage media and transitory storage media. The non-transitory storage media include, for example, a non-volatile storage medium such as an HDD (Hard Disk Drive), and ROM (Read Only Memory). The non-transitory storage media may include a portable storage medium such as a CD (Compact Disk), and a DVD (Digital Versatile Disk). Programs executed by the processor 7 are stored in a non-transitory storage medium.

Moreover, in the non-transitory storage medium constituting the memory 9 is stored an algorithm of machine learning.

The transitory storage medium is a volatile storage medium such as RAM (Random Access Memory).

The user interface 10 can accept an operator's input. For example, the user interface 10 accepts an input of a command and/or information from a user. The user interface 10 is adapted to include a keyboard, hard keys, a trackball, a rotary control, soft keys, and the like. The user interface 10 may include a touch screen that displays soft keys and the like.

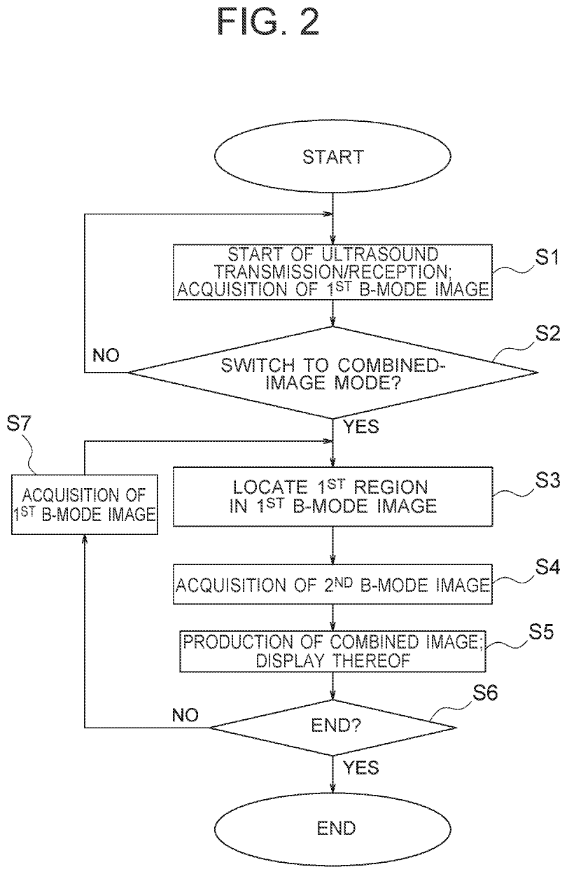

Next, an operation of the ultrasonic diagnostic apparatus in the present embodiment will be described based on the flow chart shown in FIG. 2. As an example, processing for displaying a real-time image will be described here. First, at Step S1, the processor 7 controls the ultrasonic probe 2 to start ultrasound transmission/reception to/from a patient. Ultrasound transmitted at Step S1 will be referred to herein as first ultrasound. The processor 7 produces a first B-mode image BI1 based on echo signals resulting from transmission of the first ultrasound, and displays the image on the display 8. In the present embodiment, ultrasound transmission/reception is performed on, for example, a breast of the patient. However, it will be easily recognized that the present invention is not limited to the breast as an object.

Note that the first B-mode image BI1 and a second B-mode image B12 described later include contrast-enhanced images.

The first B-mode image BI1 is obtained using a first condition group. The first condition group is comprised of several kinds of parameters used for obtaining the first B-mode image BI1, and includes a plurality of conditions. For example, the first condition group includes first transmit conditions for transmitting first ultrasound. The first condition group also includes first processing conditions for echo signals for the first ultrasound, and for raw data and image data based on the echo signals for the first ultrasound. Therefore, at Step S1, the first ultrasound is transmitted using the first transmit conditions, and the first B-mode image BI1 based on echo signals for the first ultrasound is produced by processing using the first processing conditions.

The first transmit conditions include a transmit frequency. The first transmit conditions may also include a deflection angle for an acoustic line of transmitted ultrasound when performing compounding processing. Specifically, the processor 7 may transmit, as the first ultrasound, ultrasound in a plurality of frames each having a different acoustic-line direction on a frame-by-frame basis, generate data for a B-mode image in a plurality of frames based on the resulting echo signals, and add the data for the B-mode image over the plurality of frames together to obtain the first B-mode image BI1 (compound image) in one frame.

The first processing conditions include receive conditions including a receive frequency, a magnitude of gain, a function used in logarithm compression, and an algorithm for adding data for a B-mode image over a plurality of frames. Therefore, processing using the first processing conditions includes amplification processing with a first gain, filter processing with a first receive frequency, logarithm compression processing using a first function, data addition processing for a B-mode image over a plurality of frames using a first algorithm, and the like. For example, the amplification processing may be performed on analog echo signals at the receiver 5. The filter processing and logarithm compression processing are performed on, for example, raw data by the processor 7. Moreover, the data addition processing is processing of adding data for the latest frame and those for frames displayed before the latest frame, and is performed by the processor 7. The data addition processing is performed on raw data or image data. However, it will be easily recognized that the processing cited herein is exemplary, and still other processing may be performed to produce the first B-mode image BI1.

The first condition group is defined to achieve brightness ensuring visibility in regions (those other than a first region R1, which will be discussed later) free from an acoustic shadow, which will be discussed later, in the first B-mode image BI1.

Next, at Step S2, the processor 7 decides whether or not to switch the mode to a combined-image mode for displaying a combined image. Once the user interface 10 has accepted an operator's input for switching to the combined-image mode, the processor 7 decides to switch the mode to the combined-image mode (“Yes” at Step S2), and the flow goes to Step S3. In the case that a decision is made not to switch to the combined-image mode (“No” at Step S2), the flow goes back to Step S1, and the first B-mode image BI1 in a new frame is displayed.

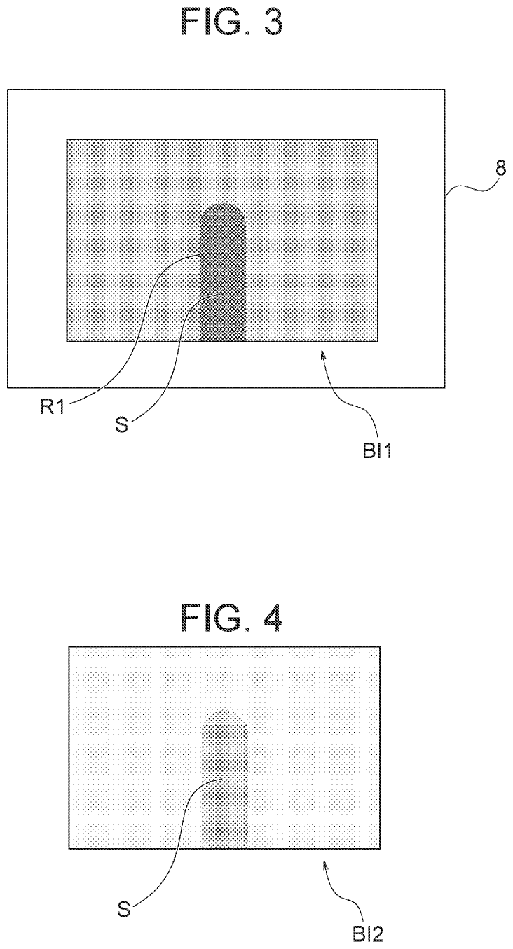

In the case that an acoustic shadow S appears in the first B-mode image BI1 displayed on display 8 as shown in FIG. 3, the operator performs an input for commanding switching to the combined-image mode at the user interface 10. The acoustic shadow S appears by an effect of ultrasound attenuation in a tumor mass, for example. The brightness in the acoustic shadow S is insufficient to observe the presence/absence of a rupture in a posterior boundary between a mammary gland and a pectoralis major muscle, or the presence/absence of a lesion in the inside of a massive tumor mass.

Next, at Step S3, the processor 7 locates a first region R1 having lower brightness than surrounding regions in the first B-mode image BI1. For example, the processor 7 detects an outline of a region having lower brightness than a required brightness TH in the first B-mode image BI1 based on data of the first B-mode image BI1, and locates the region enclosed by the outline as first region R1. In the present embodiment, the required brightness TH is set to a value allowing detection of the acoustic shadow S as the first region R1. That is, in the present embodiment, the first region R1 is a region of the acoustic shadow S.

The processor 7 may locate the first region R1 based on a signal from the user interface 10, rather than based on the data of the first B-mode image BI1. Specifically, once the user interface 10 has accepted an operator's input indicating the outline of the first region R1 in the first B-mode image BI1 displayed on the display 8, the processor 7 locates the first region R1 in the first B-mode image BI1 based on the operator's input at the user interface 10. The operator performs an input indicating the outline of the acoustic shadow S at the user interface 10.

Next, at Step S4, the processor 7 acquires a second B-mode image BI2 shown in FIG. 4. Note that it is not always necessary to display the second B-mode image BI2 on the display 8.

The second B-mode image BI2 is obtained using a second condition group. The second condition group is comprised of several kinds of parameters used for obtaining the second B-mode image BI2, and includes a plurality of conditions. For example, the second condition group includes second transmit conditions that are transmit conditions for transmitting second ultrasound and have different properties from those of the first transmit conditions. The second condition group may also include second processing conditions that are processing conditions for obtaining the second B-mode image BI2 and have different properties from those of the first processing conditions. The second condition group may include the same processing conditions as the first processing conditions.

Similarly to the first transmit conditions, the second transmit conditions include a transmit frequency, and a deflection angle for an acoustic line of transmitted ultrasound. Moreover, similarly to the first processing conditions, the second processing conditions include receive conditions including a receive frequency, a magnitude of gain, a function used in logarithm compression, and a data addition algorithm for a B-mode image over a plurality of frames. The second condition group including these conditions includes a condition(s) with a different property from that of at least one of the plurality of conditions included in the first condition group. By the expression ‘a different property’ is meant that a parameter value or an algorithm for a condition included in the second condition group is different from that for a condition included in the first condition group. The second condition group is set so that the second B-mode image BI2 has higher brightness than the first B-mode image BI1. More specifically, the second condition group is set so that visibility of the acoustic shadow S in the second B-mode image BI2 is improved relative to that in the first B-mode image BI1, and the second B-mode image BI2 has brightness to allow observation of the presence/absence of a rupture in the posterior boundary in the acoustic shadow S and/or the presence/absence of a lesion in the inside of a massive tumor mass.

At Step S4, the processor 7 may transmit new second ultrasound from the ultrasonic probe 2 using the second transmit conditions. In this case, the second ultrasound may be transmitted to a region having the same size as the first ultrasound. In transmitting the second ultrasound, the processor 7 may perform processing using processing conditions with the same properties as the first processing conditions or using the second processing conditions on echo signals for the second ultrasound acquired from the same region as the first ultrasound, and on raw data and image data based on the echo signals for the second ultrasound, and produce the second B-mode image BI2 for the same region in which the first B-mode image BI1 is produced. In other words, the second B-mode image B12 may be an image obtained using the second transmit conditions and the same processing conditions as the first processing conditions or an image obtained using the second transmit conditions and the second processing conditions.

Moreover, the processor 7 may produce the second B-mode image B12 without performing new transmission/reception, and with performing processing using the second processing conditions on the echo signals for the first ultrasound obtained at Step S1, and on the raw data and image data for the first B-mode image BI. In other words, the second B-mode image B12 may be an image obtained using the same transmit conditions as the first transmit conditions and the second processing conditions.

The acquisition of the second B-mode image BI2 will be described in more detail hereinbelow. For example, the processor 7 transmits second ultrasound having a lower transmit frequency than the first ultrasound from the ultrasonic probe 2, and produces a second B-mode image BI2 based on the resulting echo signals. Alternatively, the processor 7 may produce the second B-mode image BI2 by transmitting second ultrasound from the ultrasonic probe 2 so that the deflection angle of an acoustic line in the second ultrasound is greater than that in the first ultrasound, and performing compounding processing.

Moreover, the processor 7 may produce the second B-mode image BI2 by performing amplification processing on echo signals for the first ultrasound or those for the second ultrasound with a second gain greater than the first gain. Furthermore, the processor 7 may produce the second B-mode image BI2 by performing filter processing on raw data obtained from the echo signals for the first ultrasound or those for the second ultrasound with a second receive frequency lower than the first receive frequency. Note that in the case that the second ultrasound having a lower transmit frequency than the first ultrasound is transmitted as described above, it is desirable to perform filter processing with a second receive frequency lower than the first receive frequency.

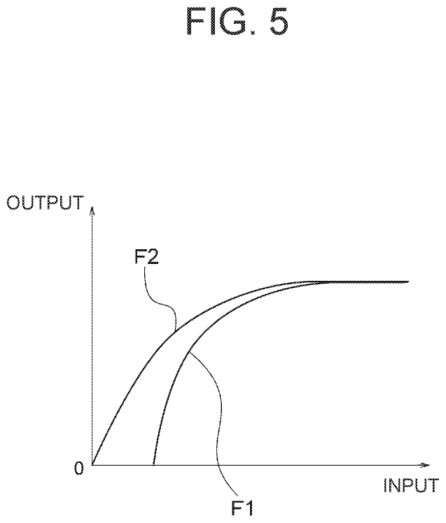

Moreover, the processor 7 may produce the second B-mode image BI2 after performing logarithm compression processing on the raw data obtained from the echo signals for the first ultrasound or those for the second ultrasound using a second function different from a first function. FIG. 5 shows examples of the first function F1 and second function F2. As compared with the first function, the second function F2 has output data of greater value for a smaller value of input data.

In addition, the processor 7 may produce the second B-mode image B12 in one frame by performing addition processing on the data of the first B-mode image BI1 over a plurality of frames using a second algorithm different from the first algorithm. Alternatively, the processor 7 may produce the second B-mode image B12 in one frame by performing addition processing using the second algorithm on the data of the second B-mode image data BI2 over a plurality of frames obtained from echo signals for the second ultrasound. The data of the first B-mode image BI1 and the data of the second B-mode image BI2 may be raw data or image data.

For example, the second algorithm is an algorithm with which a structure in a low-brightness region is more enhanced by increasing a weight on the first B-mode image BI1 in a frame temporally earlier than the first B-mode image BI1 in the latest frame or on the second B-mode image BI2 in a frame temporally earlier than the second B-mode image BI2 in the latest frame.

The processor 7 may set the second condition group so that a B-mode image BI2 with more appropriate brightness can be obtained for a region corresponding to the first region R1 based on the condition regarding brightness of the image in the first region R1 in the first B-mode image BI1 or the like. The expression ‘more appropriate brightness’ refers to brightness that allows observation of the presence/absence of a rupture in the posterior boundary between the mammary gland and pectoralis major muscle, or the presence/absence of a lesion in the inside of a massive tumor mass. The second condition group defined based on the image in the first region R1 may be stored beforehand in the memory 9. In this case, a correspondence between a state regarding brightness of the image in the first region R1 and a second condition group defined depending upon the state may be identified according to experimentation, an empirical rule, and/or the like, and stored in the memory 9 beforehand. Moreover, the processor 7 may be adapted to present on the display 8 a plurality of candidates of the second condition group according to the condition regarding brightness of the image in the first region R1. In this case, one second condition group is defined by selection by the operator at the user interface 10.

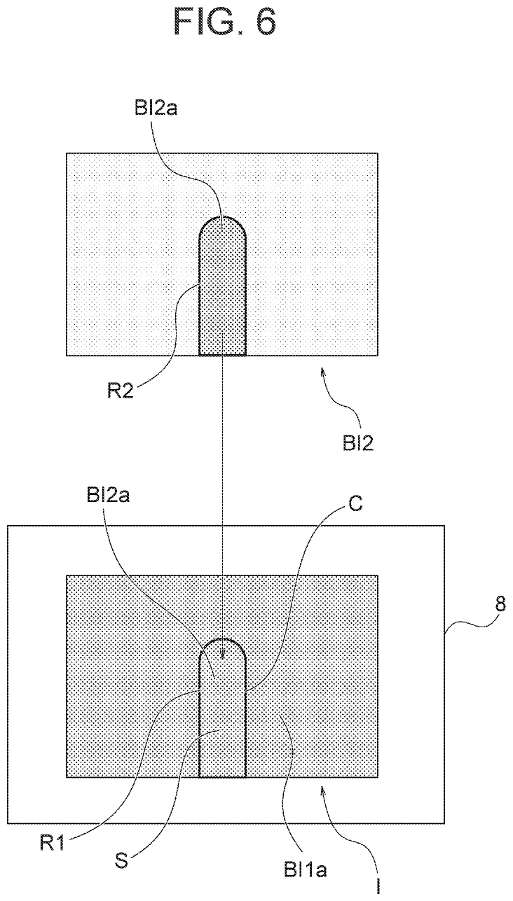

Once the second B-mode image BI2 has been obtained at Step S4, the processor 7 produces a combined image I, and displays it on the display 8 at Step S5, as shown in FIG. 6. Specifically, the processor 7 produces a combined image I in which an image BI2a in the second region R2 in the second B-mode image B12 at a position corresponding to the position of the first region R1 is combined into the position of the first region R1 in the first B-mode image BI1, and displays the combined image I on the display. Since in the combined image I, the portion in the first B-mode image BI1 in which the acoustic shadow S has appeared is thus replaced by the image BI2a in the second region R2 in the second B-mode image B12, visibility in the acoustic shadow S is improved.

The image in regions other than the first region R1 in the combined image I is an image BI1a in regions other than the first region R1 in the first B-mode image BI1. Hence, a high-brightness region surrounding the acoustic shadow S in the first B-mode image BI1 is still displayed as is in the combined image I. Therefore, visibility is maintained in the high-brightness region surrounding the acoustic shadow S.

The processor 7 may display on the display 8 at least one of a text, a geometrical figure, and an image indicating that the combined image I is being displayed. In FIG. 6, the processor 7 displays on the display 8 a contour line C representing an outline of the image BI2a displayed in the first region R1 in the combined image I as the geometrical figure indicating that the combined image I is being displayed.

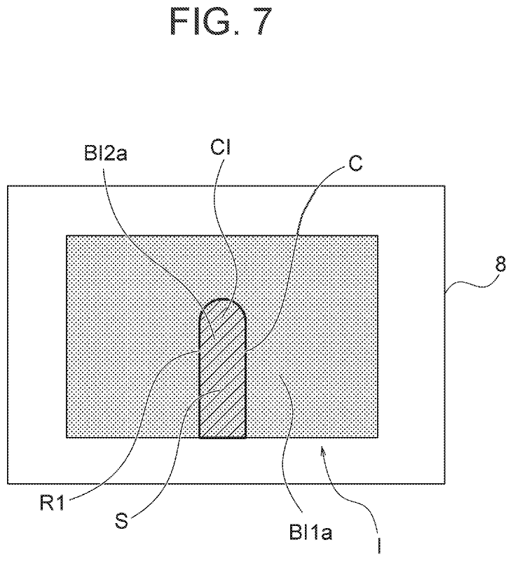

Moreover, as the image indicating that the combined image I is being displayed, the processor 7 may display on the display 8 a color image CI having a required degree of transparency against the background of the image BI2a in the first region R1 in the combined image I, as shown in FIG. 7. In FIG. 7, the color image CI is represented by hatching. As shown in FIG. 7, the color image CI may be displayed along with the contour line C. Alternatively, the color image CI may be displayed without displaying the contour line C.

Furthermore, as the image indicating that the combined image I is being displayed, the processor 7 may display on the display 8 a degraded-resolution version of the image BI1a in the combined image I, although not particularly shown. It should be noted that the degree of degradation of resolution of the image BI1a should be such one that it does no harm to diagnosis, etc.

Once the combined image I has been displayed at Step S5, the flow goes to Step S6. At Step S6, once a signal indicating acceptance of an operator's input for terminating the processing has been input from the user interface 10, the processor 7 decides to terminate the processing (“Yes” at Step S6).

On the other hand, in the case that a decision is made not to terminate the processing at Step S6 (“No” at Step S6), the flow goes to Step S7. At Step S7, first ultrasound is transmitted, and a first B-mode image BI1 in a new frame is produced and displayed, as in Step S1. Once the first B-mode image BI1 has been obtained at Step S7, the processing at and after Step S3 is executed again to obtain a combined image I in the new frame for display.

Next, a variation of the embodiment will be described. First, a first variation will be described. The processor 7 may produce the second B-mode image B12 only in a region corresponding to the first region R1 that is smaller than the region in a patient for which the first B-mode image BI1 is produced.

Next, a second variation will be described. The processor 7 may decide whether or not to acquire the second B-mode image BI2 based on the state of brightness, etc. of the first B-mode image BI1 in the first region R1. In the case that a decision is made not to acquire the second B-mode image BI, the first B-mode image BI1 in a new frame is acquired.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive. For example, the processing in the present invention may be similarly applied to a case in which images are displayed off-line, rather than a case in which real-time images are displayed, that is, a case in which images based on raw data or image data stored in the memory 9 are displayed. In this case, data of the second B-mode image B12 is acquired based on data of the first B-mode image BI1 stored in the memory 9, and a combined image I is obtained.

Moreover, the present invention may be applied to other regions, such as an orthopedic region, in addition to the mammary gland region.

Furthermore, the embodiment described above may be a method of controlling an ultrasonic diagnostic apparatus, said apparatus comprising: an ultrasonic probe for transmitting ultrasound to a patient and receiving echo signals for said ultrasound; one or more processors; and a display, wherein said one or more processors execute the steps of:

controlling said ultrasonic probe to transmit said ultrasound,

producing a first B-mode image and a second B-mode image based on echo signals for said ultrasound, said first B-mode image being obtained using a first condition group including a plurality of conditions, said second B-mode image being obtained using a second condition group including a plurality of conditions, said second condition group including a condition(s) with a property different from that of at least one of the plurality of conditions included in said first condition group, said second condition group being defined so that brightness of said second B-mode image is higher than that of said first B-mode image,

locating a first region in said first B-mode image having lower brightness than a surrounding region, and

producing a combined image in which an image of a second region at a position in said second B-mode image corresponding to a position of said first region is combined into the position of said first region in said first B-mode image for display on said display.

1. An ultrasonic diagnostic apparatus comprising:
an ultrasonic probe for transmitting ultrasound to a patient and receiving echo signals for said ultrasound;
one or more processors; and
a display, wherein said one or more processors are configured to:
control said ultrasonic probe to transmit said ultrasound,
produce a first B-mode image and a second B-mode image based on echo signals for said ultrasound, said first B-mode image being obtained using a first condition group including a plurality of conditions, said second B-mode image being obtained using a second condition group including a plurality of conditions, said second condition group including a condition(s) with a property different from that of at least one of the plurality of conditions included in said first condition group, said second condition group being defined so that brightness of said second B-mode image is higher than that of said first B-mode image,
locate a first region in said first B-mode image having lower brightness than a surrounding region,
locate a second region at a position in said second B-mode image corresponding to a position of said first region,
combine said second B-mode image of said second region into the position of said first region in said first B-mode image to produce a combined image, and
display said combined image on said display.

2. The ultrasonic diagnostic apparatus as recited in claim 1, wherein said one or more processors are configured to:
transmit, as said ultrasound, first ultrasound from said ultrasonic probe using first transmit conditions included in said first condition group, and produce, as said first B-mode image based on said echo signals, an image based on the echo signals for said first ultrasound received by said ultrasonic probe by performing processing using first processing conditions included in said first condition group, and
transmit, as said ultrasound, second ultrasound from said ultrasonic probe using second transmit conditions included in said second condition group, said second transmit conditions having different properties from those of said first transmit conditions, and produce, as said second B-mode image based on said echo signals, an image based on the echo signals for said second ultrasound received by said ultrasonic probe by performing processing using processing conditions included in said second condition group, said processing conditions having identical properties to those of said first processing conditions, or processing using second processing conditions included in said second condition group, said second processing conditions having different properties from those of said first processing conditions.

3. The ultrasonic diagnostic apparatus as recited in claim 1, wherein said one or more processors are configured to:
transmit, as said ultrasound, first ultrasound from said ultrasonic probe using first transmit conditions included in said first condition group, and produce, as said first B-mode image based on said echo signals, an image based on the echo signals for said first ultrasound received by said ultrasonic probe by performing processing using first processing conditions included in said first condition group, and
produce, as said second B-mode image based on said echo signals, an image based on the echo signals for said first ultrasound by performing processing using second processing conditions included in said second condition group, said second processing conditions having different properties from those of said first processing conditions.

4. The ultrasonic diagnostic apparatus as recited in claim 1, wherein: said one or more processors locate, as said first region, a region in said first B-mode image having lower brightness than required brightness based on data of said first B-mode image.

5. The ultrasonic diagnostic apparatus as recited in claim 1, further comprising:
a user interface for accepting an operator's input, wherein
said one or more processors are further configured to display said first B-mode image on said display, and once said user interface has accepted an operator's input indicating said first region in said first B-mode image displayed on said display, said one or more processors locate said first region based on said operator's input at said user interface.

6. The ultrasonic diagnostic apparatus as recited in claim 1, wherein: said one or more processors display on said display at least one of a text, a geometrical figure, and an image indicating that said combined image is being displayed.

7. The ultrasonic diagnostic apparatus as recited in claim 6, wherein: said one or more processors display, as said geometrical figure on said display, a contour line representing an outline of said second B-mode image displayed in said first region in said combined image.

8. The ultrasonic diagnostic apparatus as recited in claim 6, wherein: said one or more processors display, as said image on said display, a color image having a required degree of transparency in said first region in said combined image against a background of said second B-mode image.

9. The ultrasonic diagnostic apparatus as recited in claim 6, wherein: said one or more processors display, as said image on said display, a degraded-resolution version of said first image in said combined image.

10. The ultrasonic diagnostic apparatus as recited in claim 1, wherein: said one or more processors produce said second B-mode image for a region in said patient identical to the region for which said first B-mode image is produced, or for a region in said patient corresponding to said first region.

11. A method of controlling an ultrasonic diagnostic apparatus, said apparatus comprising: an ultrasonic probe for transmitting ultrasound to a patient and receiving echo signals for said ultrasound; one or more processors; and a display, wherein said one or more processors execute the processing of:
controlling said ultrasonic probe to transmit said ultrasound,
producing a first B-mode image and a second B-mode image based on echo signals for said ultrasound, said first B-mode image being obtained using a first condition group including a plurality of conditions, said second B-mode image being obtained using a second condition group including a plurality of conditions, said second condition group including a condition(s) with a property different from that of at least one of the plurality of conditions included in said first condition group, said second condition group being defined so that brightness of said second B-mode image is higher than that of said first B-mode image,
locating a first region in said first B-mode image having lower brightness than a surrounding region,
locating a second region at a position in said second B-mode image corresponding to a position of said first region,
combining said second B-mode image of said second region into the position of said first region in said first B-mode image to produce a combined image, and
displaying said combined image on said display.

12. The method of controlling said ultrasonic diagnostic apparatus as recited in claim 11, wherein said one or more processors execute the processing of:
transmitting, as said ultrasound, first ultrasound from said ultrasonic probe using first transmit conditions included in said first condition group, and producing, as said first B-mode image based on said echo signals, an image based on the echo signals for said first ultrasound received by said ultrasonic probe by performing processing using first processing conditions included in said first condition group, and
transmitting, as said ultrasound, second ultrasound from said ultrasonic probe using second transmit conditions included in said second condition group, said second transmit conditions having different properties from those of said first transmit conditions, and producing, as said second B-mode image based on said echo signals, an image based on the echo signals for said second ultrasound received by said ultrasonic probe by performing processing using processing conditions included in said second condition group, said processing conditions having identical properties to those of said first processing conditions, or processing using second processing conditions included in said second condition group, said second processing conditions having different properties from those of said first processing conditions.

13. The method of controlling said ultrasonic diagnostic apparatus as recited in claim 11, wherein said one or more processors execute the processing of:
transmitting, as said ultrasound, first ultrasound from said ultrasonic probe using first transmit conditions included in said first condition group, and producing, as said first B-mode image based on said echo signals, an image based on the echo signals for said first ultrasound received by said ultrasonic probe by performing processing using first processing conditions included in said first condition group, and
producing, as said second B-mode image based on said echo signals, an image based on the echo signals for said first ultrasound by performing processing using second processing conditions included in said second condition group, said second processing conditions having different properties from those of said first processing conditions.

14. The method of controlling said ultrasonic diagnostic apparatus as recited in claim 11, wherein: said one or more processors execute the processing of locating, as said first region, a region in said first B-mode image having lower brightness than required brightness based on data of said first B-mode image.

15. The method of controlling said ultrasonic diagnostic apparatus as recited in claim 11, said ultrasonic diagnostic apparatus further comprising a user interface for accepting an operator's input, wherein
said one or more processors are further execute the processing of displaying said first B-mode image on said display, and once said user interface has accepted an operator's input indicating said first region in said first B-mode image displayed on said display, said one or more processors locate said first region based on said operator's input at said user interface.

16. The method of controlling said ultrasonic diagnostic apparatus as recited in claim 11, wherein: said one or more processors execute the processing of displaying on said display at least one of a text, a geometrical figure, and an image indicating that said combined image is being displayed.

17. The method of controlling said ultrasonic diagnostic apparatus as recited in claim 16, wherein: said one or more processors execute displaying, as said geometrical figure on said display, a contour line representing an outline of said second B-mode image displayed in said first region in said combined image.

18. The method of controlling said ultrasonic diagnostic apparatus as recited in claim 16, wherein: said one or more processors execute the processing of displaying, as said image on said display, a color image having a required degree of transparency in said first region in said combined image against a background of said second B-mode image.

19. The method of controlling said ultrasonic diagnostic apparatus as recited in claim 16, wherein: said one or more processors execute the processing of displaying, as said image on said display, a degraded-resolution version of said first image in said combined image.

20. The method of controlling said ultrasonic diagnostic apparatus as recited in claim 11, wherein: said one or more processors execute the processing of producing said second B-mode image for a region in said patient identical to the region for which said first B-mode image is produced, or for a region in said patient corresponding to said first region.
